(19) United States
(12) Patent Application Publication
(10) Pub. No.: US 2024/0194315 A1
(43) Pub. Date: Jun. 13, 2024

(54) Questionnaire Based Medical Billing Platform Automating ICD-10 Codes with integration of proper CPT coding

(71) Applicant: John P. Poovey, Grand Junction, CO (US)

(72) Inventor: John P. Poovey, Grand Junction, CO (US)

(21) Appl. No.: 18/532,994

(22) Filed: Dec. 7, 2023

Related U.S. Application Data
(60) Provisional application No. 63/430,965, filed on Dec. 7, 2022.

(51) Int. Cl. G16H 15/00; G06Q 30/04

(52) U.S. Cl. CPC G16H 15/00; G06Q 30/04; G06Q 40/08

(57) ABSTRACT

An AI supported questionnaire based billing platform and associated methodology includes inputting a patient health history; generating automatically a list of ICD codes based upon the patient health history; generating automatically a listing of CPT codes associated with the list ICD codes; selecting by a medical practitioner CPT codes to be performed on the patient from the CPT codes listing; generating an on-line user fillable operative notes template based upon the selected CPT codes, wherein the selected CPT codes are matched with ICD codes; prompting the practitioner to fill out all relevant portions of the operative notes template; obtaining a signature of the practitioner on the operative notes template, wherein the practitioner is prevented completing the template until all relevant portions of the operative notes template have been properly filled out; and generating a bill based upon the completed and signed operative notes template.

### RELATED APPLICATIONS

This application claims priority to U.S. Provisional patent application Ser. No. 63/430,965 filed Dec. 7, 2022, titled “Questionnaire Based Medical Billing Platform Automating ICD-10 Codes with integration of proper CPT coding” which application is incorporated herein by reference.

### BACKGROUND OF THE INVENTION

### Field of the Invention

The present invention which relates to medical billing platforms automating ICD Codes with integration of proper CPT coding.

### Background

Getting sick can be costly. That's why most people have acquired medical insurance or health insurance, including Medicare and Medicaid, for themselves and their families. With health insurance, people (theoretically) obtain a cheaper and more convenient way to get their ailments treated.

Medicare is a government national health insurance program in the United States, begun in 1965 under the Social Security Administration and now administered by the Centers for Medicare and Medicaid Services. It, primarily, provides health insurance for Americans aged 65 and older, but also for some younger people with disability status as determined by the Social Security Administration. Medicaid in the United States is a federal and state program that helps with healthcare costs for some people with limited income and resources. Medicaid also offers benefits not normally covered by Medicare, including nursing home care and personal care services. The main difference between the two programs is that Medicaid covers healthcare costs for people with low incomes while Medicare provides health coverage for the elderly. There are also dual health plans for people who have both Medicaid and Medicare. The Health Insurance Association of America describes Medicaid as “a government insurance program for persons of all ages whose income and resources are insufficient to pay for health care.”

As of 2020 it had been estimated that over 91% of the U.S. population is covered by private or public health insurance. According to United States Census Borough, in 2022 over 92% of people, or 304.0 million, had health insurance at some point during the year. Thus, not only are the patients and the doctors involved in the treatment and the diagnosis and treatment of a medical problem, but so too are the medical insurers.

In order to hasten the payment process, to document the diagnosis and treatment, and to make everyone understand the medical problem, coding systems have been developed. There are three major coding systems used by medical billers and insurers: the Current Procedural Terminology (CPT) and International Classification of Diseases (ICD) and Healthcare Common Procedure Coding System (HCPCS level II).

### Coding Systems

The American Medical Association (AMA) was actually the first to tackle the problem before HCPCS coding. In efforts to standardize reporting of medical, surgical, and diagnostic services and procedures, the association created a coding system and introduced CPT in 1966. The CPT codes (also referenced as HCPCS level 1) were and are developed by the AMA and generally are for reporting medical procedures and services such as diagnostic, laboratory, radiology, and surgical. The CPT codes describe what was done to the patient during the consultation. The procedure code listed by the practitioner generally is found by the medical provider in “the CPT book” or now via an online listing. The selected code is intended to describe the medical services and procedures done by the medical professional. It aims at providing a uniform language to describe the treatment and diagnostic procedures performed and aid in the communication between doctors, patients, and insurance companies. Most commercial payers, based on the plan design, require one or more CPT codes in any claim.

Even at this time, the government had already become a major payer of healthcare services. While it too needed to standardize healthcare claims, it also bore the responsibility of controlling costs for taxpayers. With this dual agenda, it created the HCFA Common Procedure Coding System (HCPCS). The history of HCPCS coding began in 1978 when the federal government created this coding system to attempt standardize the reporting of medical services to the federal government for reimbursement. The HCPCS system, however, underwent several changes before adoption by commercial payers, which action was eventually mandated by the Health Insurance Portability and Accountability Act (HIPAA) in 1996. Prior to the advent of procedure coding, providers submitted written descriptions of the services they performed to payers for reimbursement. This proved inefficient, in that 100 providers could report the same service with 100 different descriptions.

Standardization in medical reporting was not achieved by the development of initial coding systems as in the subsequent decade, more than 120 different coding systems came into play, causing widespread variations in payers' guidelines and claim forms. However in 1983, the HCFA Common Procedure Coding System (HCPCS) was merged with AMA's CPT system and use of the CPT system was mandated for all Medicare billing. Currently, HCPCS level 1 is the CPT system.

The two organizations collaborated on the development of a new code set to report medical-related expenses not represented in the CPT codes, items such as orthotic and prosthetic procedures, hearing and vision services, ambulance services, medical and surgical supplies, drugs, nutrition therapy, durable medical equipment, outpatient hospital care, and Medicaid. The resulting code set, also implemented in 1983, begins where the CPT coding ends. As such, it is the second of two principal subsystems of HCPCS, aptly named HCPCS Level II. Through the evolution of HCPCS coding, the CPT coding is incorporated as the backbone of Level I, and the newer HCPCS Level II is now known as a procedure coding system.

HCPCS Level II codes are maintained and updated by the Centers for Medicare & Medicaid Services under the Department of Health and Human Services, except for dentistry. HCPCS Level II Dental codes, or D codes, are a separate category of national HCPCS Level II codes known as the Current Dental Terminology (CDT) which is published, copyright protected, and licensed by the American Dental Association (ADA). The CDT lists codes for billing for dental procedures and supplies. While the CDT codes are considered HCPCS Level II codes, decisions regarding the revision, deletion, or addition of CDT codes are made by the ADA, not CMS

ICD codes, or International Classification of Disease codes, are used to describe the client's diagnosis. In other words, they refer to the specific condition that's being treated. ICD codes are developed by the World Health Organization (WHO), and they're used worldwide. In addition to being used in the process of health care billing, ICD codes are also used to identify health and disease trends across countries. Simply put, they're the standard diagnostic language used by health care providers, payers, researchers, and global public health officials.

With the distinct coding systems maintained by different parties it can be difficult to keep the systems distinct. A crude explanation is as follows:
CPT codes: Define what the provider did.
HCPCS (level II) codes: Define what the provider used.
ICD-10-CM: Define Why the provider did what was done and used what was used.

As a representative example, if a urologist diagnoses a patient with bladder cancer and performs a bladder instillation of 1 mg of Bacillus Calmette-Guerin (BCG) to treat the tumor, the medical coder might assign:
CPT codes (did): 51720 Bladder instillation of anticarcinogenic agent (including retention time)
HCPCS Level II code (used): J9030 BCG live intravesical instillation, 1 mg
ICD-10-CM code (why): C67.9 Malignant neoplasm of bladder, unspecified

Making a complex set of interacting systems more complex is there are some exceptions to these general code set concepts.

Updates to ICD codes are published on an irregular basis, with minor updates published every one to four years. Entirely new versions of the ICD, which typically include more significant changes in coding and structure, are published far less often. The current version, ICD-10, entered common use in 1994. The ICD-11 was formally adopted in May 2019 and went into effect on Jan. 1, 2022.

The CPT book is published by the American Medical Association and has 7,800 codes. The ICD book is published by the World Health Organization and has more than 200,000 codes for its ICD-10. The CPT book has a health care common procedure coding system and focuses on medications and equipment for which a patient is billed. This system is not found in the ICD book. As noted above HCPCS Level II codes are maintained and updated by the Centers for Medicare & Medicaid Services under the Department of Health and Human Services, except for dentistry which are updated and maintained by the ADA.

Practitioners are instructed generally to “look up” the relevant ICD code, which can be found online in various formats. There are also a number of websites that index ICD codes in different ways, aiming to make it easier to find the code the practitioner needs. As evident by the various indexed listings it is not always easy for practitioners to find the relevant ICD codes. This difficulty can be more pronounced in select fields such as dentistry, as dentists often have less exposure to certain ICD codes.

CPT codes must also be “looked up” by the practitioner although in select fields these can sometimes be found in practice management software. Having the correct CPT code, however is only part of the process. Consider, for example, the dental field wherein Dental CPT codes are used to report procedures to medical payers for dental works. As discussed above, this code set is maintained by the American Medical Association as compared with the HCPCS (level II) for dentistry that are maintained by the ADA. Practitioners are instructed that “it is important to follow the dental claim form instructions exactly when submitting a medical claim. Common claim form errors include, but are not limited to, the use of punctuation, the absence of a description when reporting an unlisted CPT code, and use of the appropriate modifier or qualifier, when required.” Dentists currently have to invest in staff training and resources to ensure accurate completion of any claim form to all third parties. It is noted that Dentists will typically be more familiar with the separate Current Dental Terminology (CDT) (HCPCS Level II dental codes) which have been created, published, and kept up-to-date by the American Dental Association. The CDT has been primarily prepared to cover all the dental procedures and examinations in billing and coding.

It is time consuming for any medical practitioner to keep abreast of all the separate coding systems maintained by separate entities. This becomes even more apparent in a specialty such as dentistry that introduces yet another coding entity and code set. Medical practitioners will often become familiar with the coding of their particular niche, but this can actually detrimentally effect the patient care as practices may be less willing to step outside of billing areas in which they are familiar. Having a claim rejected may mean the practitioner is simply not compensated for the alleged “miss-coded procedure”, and such are less likely to be offered by the practitioner in the future.

The patent literature illustrates platforms attempting to address the problems with the prior art. For example U.S. Pat. No. 11,361,853, developed by Practice Velocity, LLC, discloses a method and system for automated medical records processing with telemedicine. The method and system includes plural electronic medical templates specifically designed such that they reduce the complexity and risk associated with collecting virtual patient encounter information, creating a medical diagnosis, tracking the patient through the medical processes during a telemedicine session and generate the appropriate number and type medical codes for a specific type of medical practice when processed. The medical codes and other types of processed virtual patient encounter information are displayed in real-time on electronic medical records and invoices immediately after a virtual patient encounter from a telemedicine visit. See a related system by Practice Velocity, LLC, implementing patient tracking in U.S. Pat. Nos. 10,714,213 and 9,842,188. See also U.S. Pat. No. 8,606,594 by Practice Velocity, LLC for automated medical records processing.

U.S. Pat. No. 11,024,424, developed by Nuance Communications, Inc, discloses a computer assisted coding system which according to some aspects, a system for automatically processing text comprising information regarding a patient encounter to assign medical codes to the text is provided. The system comprises at least one storage medium storing processor-executable instructions, and at least one processor configured to execute the processor-executable instructions to perform analyzing the text to extract a plurality of facts from the text, identifying at least one of the plurality of facts to be excluded from consideration when assigning medical codes to the text, and evaluating each of the plurality of facts, except for the identified at least one fact, to assign one or more medical codes to the text. Also Nuance Communications, Inc, in U.S. Pat. No. 10,366,642 discloses techniques for medical coding include applying a natural language understanding (NLU) engine to a free-form text documenting a clinical patient encounter, to derive a first set of one or more medical billing codes for the clinical patient encounter and a link between each code in the first set and a corresponding portion of the free-form text. The first set of codes may be compared to a second set of one or more medical billing codes approved by one or more human users for the patient encounter, to identify at least one code in the first set that overlaps with at least one code in the second set. The code in the second set approved by the one or more human users may be retained instead of the overlapping code in the first set derived by the NLU engine. Further Nuance Communications, Inc, in U.S. Pat. No. 10,319,004, 9,916,420 and 9,679,107 disclose a medical documentation system and a CDI system may be linked together, or integrated, so there is a tie between the two systems that allows for a much more efficient and effective CDI process. In one disclosed embodiment, a CDI system receives from a medical documentation system a structured data set including at least some information relating to one or more medical facts the medical documentation system automatically extracted from text documenting a patient encounter. Additionally Nuance Communications, Inc, in U.S. Patent Application Publication 2020-0126643 Techniques are provided whereby a clarification request may be generated with a clinical documentation improvement (CDI) system for resolution by a clinician, and notification of the clarification request may be transmitted to a medical coding system. At a medical coding system, notification may be received of a clarification request generated at a CDI system for resolution by a clinician. In some embodiments, the medical coding system may be a computer-assisted coding (CAC) system.

Passport Health Communications, Inc., in U.S. Pat. No. 10,275,576 discloses an automatic medical coding system that parses features of natural language diagnosis and procedure information. The features are compared to elements of a medical coding system. Medical codes corresponding medical coding system elements that match features of the diagnosis and procedure information are mapped to the received diagnosis and procedure information. The mapped medical code is assigned a score reflecting the estimated reliability of the mapped medical code based on the amount of manipulation of the received diagnosis and procedure information leading to the match. The scored medical code may be submitted to a workflow making use of medical codes. The scored medical code may optionally be presented to a user for review prior to further utilization of the scored medical code.

Greenway Medical Technologies, Inc. in U.S. Pat. Nos. 8,781,853, 8,738,396 and 8,050,938 discloses an integrated medical software system with location-driven bill coding is disclosed. The system comprises an information component that automatically retrieves payment method data and demographic data for a patient and location data for a healthcare provider; a clinical component that captures at least one of a diagnosis code, a procedure code, and an evaluation and management (E&M) code as at least one of a clinician and a staff member at the healthcare provider inputs clinical data for the patient into the an electronic document during an encounter with the patient; a mapping component that automatically associates a billing code with the at least one of a pathology code, a procedural code, and an E&M code and assigns a service cost to the billing code based on a pre-defined fee schedule, said pre-defined fee schedule being automatically chosen based on the location data for the healthcare provider; and a service detail entry component that uses the payment method data and the demographic data for the patient and the service cost to automatically generate at least one of a bill, a claim, and a statement for the patient.

Even General Electric Company has developed relevant technology in U.S. Pat. No. 8,346,804 which discloses a method for mapping of medical code schemes includes processing a plurality of coded concepts to determine a potential match between a code from a first code scheme in the plurality of coded concepts and a code from a second code scheme in the plurality of coded concepts. The method includes assigning a probability to each potential match of a code from the first code scheme and a code from the second code scheme. The method includes generating an alphanumeric indication of the probability of each potential match between the first code scheme and the second code scheme from the plurality of coded concepts and generating a graphical representation of the plurality of coded concepts. The method includes outputting the alphanumeric indication and the graphical representation to a user and accepting user input to select a match between the first code scheme and the second code scheme.

A 3M holding company, 3M Innovative Properties Company, developed relevant technology in the vascular field in U.S. Patent Application Publication 2020-00411147 disclosing a system and method for identifying medical procedure codes and medical diagnosis codes from physician reports that describe a vascular interventional radiology procedure using a combination of natural language processing (NLP) and human medical coders. In one embodiment, the system and method of the present invention creates billing results, and or other documents, that are compliant with applicable legal and policy instructions from the government or a medical institution. Medical billing codes are efficiently extracted from medical reports using a. NLP engine and a graphical user interface optimized for understanding the VasIR medical procedure described in the report.

Mirr, Inc. in U.S. Patent Application Publication 2020-0043579 discloses a system and method for improved diagnostic and treatment of patients wherein a clinician's notes of diagnosis, treatment or testing are electronically recorded thereby becoming an entry in a patient specific EHR. Coding appropriate to diagnosis etc., may be adapted by the clinician and the coding integrated into the EHR entry. The EHR may be searched using adapted codes as an index of diagnosis, treatment and testing. The tool and method includes program for facilitating entry of clinician notes (auto complete and autocorrection) and suggestion of appropriate code entries correlated to the content of the clinician notes. The program may include authentication protocols for protecting privacy and accesses to patient EHR. Code and code descriptors may be the product of established protocols such as CBD-10 or CPT. The tool and method may allow immediate search of patient's EHR for prior relevant conditions or treatment thereby facilitating prompt treatment of underlying cause of malady.

Enlitic, Inc. in U.S. Patent Application Publication 2020-0160301 discloses a medical billing verification system can be operable to determining at least one medical code corresponding to a medical report for a patient, where the at least one medical code indicates a medical procedure that was performed on the patient a billing rate that corresponds to the at least one medical code can be determined by utilizing a mapping of medical codes to billing rates. Billing data corresponding to the medical procedure that was performed on the patient can be received, where the billing data indicates an amount that was billed for the medical procedure. Billing verification data can be generated by comparing the billing rate to the billing data. An improper billing notification can be generated for transmission to a client device via the network for display via a display device in response to the billing verification data indicating the billing rate compares unfavorably to the billing data.

The relevant prior art is not limited to the United States as Automated Clinical Guidelines, LLC in Australian Published Patent Application 2023-214261 discloses a web-based platform which guides a user's encounter with a patient and generates a medical record of the encounter. The web-based platform comprises a processor in communication with an output display device and an input user interface, and a knowledge base. The processor outputs information to the output display device, 5 accesses the knowledge base, and receives input from the input user interface. The output display device requests patient information in response to presented successive prompts for patient medical information. The prompts are responsive to the knowledge base and to prior responses to prior prompts as entered through the input user interface. The output display device presents a patient medical condition report based on responses to requests and prompts. ICD codes are included within the knowledge base and included within the patient medical condition report. The codes are determined, as applicable, as each prompt and response is entered.

The above patent literature provides a brief overview of the state of the art and the U.S. patents and published patent applications are incorporated herein by reference. However there remains a need for medical billing software that can effectively and efficiently integrate update CPT, HCPCS Level II and ICD codes.

### SUMMARY OF THE INVENTION

In summary, the questionnaire based billing platform of the present invention allows a medical practitioner, such as a dentist, to quickly generate appropriate diagnostic codes (e.g. ICD-10 codes) and collate them and allow them to obtain proper CPT codes based upon the ICD codes. In other words, the medical billing platform of the present invention facilitates medical billing for procedures deemed within the medical scope of the practice and generating billing with appropriate ICD and CPT codes that are CMS approved and audit proof. The questionnaire based billing platform creates an autofill note that may be customized by the individual medical practitioner or user for their general practice while generating essentially pre-approved final billing.

In one embodiment of the present invention a questionnaire based billing platform and associated methodology includes inputting a patient health history into the platform; generating automatically by the platform a list of one or more ICD codes based upon the patient health history; generating automatically by the platform a listing of CPT codes associated with the list of one or more ICD codes; selecting on the platform by a medical practitioner one or more CPT codes to be performed on the patient from the listing of CPT codes associated with the list of one or more ICD codes; generating an on-line user fillable operative notes template based upon the selected CPT codes, wherein the selected CPT codes are matched with ICD codes; prompting the practitioner to fill out all relevant portions of the on-line user fillable operative notes template; obtaining a signature of the practitioner on the on-line user fillable operative notes template following the practitioner filling out all relevant portions of the on-line user fillable operative notes template, wherein the practitioner is prevented from providing a signature to complete the form until all relevant portions of the on-line user fillable operative notes template have been properly filled out; and generating a bill by the platform based upon the completed and signed on-line user fillable operative notes template.

In one embodiment of the present invention an on-line questionnaire based billing platform includes an online patient health history input form for each patient on the platform; an automatically generated listing of one or more ICD codes based upon each patient health history; an automatically generated listing of CPT codes associated with the list of one or more ICD codes; select CPT codes to be performed on the patient which are selected by the medical practitioner from the listing of CPT codes associated with the list of one or more ICD codes; an on-line user fillable operative notes template generated by the platform based upon the selected CPT codes, wherein the selected CPT codes are matched with ICD codes; wherein the system is configured to prompt the medical practitioner to fill out all relevant portions of the on-line user fillable operative notes template; wherein the system is configured to obtain a signature of the medical practitioner on the on-line user fillable operative notes template following the practitioner filling out all relevant portions of the on-line user fillable operative notes template, wherein the medical practitioner is prevented from providing a signature to complete the on-line user fillable operative notes template until all relevant portions of the on-line user fillable operative notes template have been properly filled out; and a bill generated by the platform based upon the completed and signed on-line user fillable operative notes template.

Currently a medical practitioner would conduct a health history of a patient then proceed separately to look up codes that would be appropriate (via manually, on-line or various systems or even combinations of such methods) while the questionnaire based billing platform of the present invention effectively allows the health history intake to automatically generate the relevant ICD codes. The generated listing of ICD will then generate associated CPT codes which can be selected by the practitioner and once the practitioner selects the desired CPT codes (that are matched with the relevant ICD codes) which are being performed, an operative notes template will be generated that will allow the practitioner to fill in the relevant portions with the operative notes template including prompts for all required part. Following the filling of the template there is a signature requirement to complete the process. The practitioner, or their billing department, then can user completed signed notes to generate a bill that is audit proof to be paid by the insurer.

Additionally the questionnaire based billing platform of the invention allows for easy pre-approval of the patient which itself can allow the patient to make informed medical decisions based upon known coverage. The questionnaire based billing platform of the invention generates approved billing thus facilitates the third party creation of EOB (explanation of benefits), and the platform provides that such an EOB can be uploaded by practitioner for improved accounting (allowing tracking and proper accounting of differences between billable, allowable and reimbursable amounts for a given patient)

As noted, the questionnaire based billing platform of the invention more easily generates and formats bills to be audit proof and to be timely paid by insurers. Additionally in the constantly changing billing environment the platform of the invention avoids having everyone go through denial due to insurance or other regulatory agency changes. Systemic changes may be made in real time to update billing due to changes.

### BRIEF DESCRIPTION OF THE FIGURES

FIG. 1 is a schematic flow chart of questionnaire based medical billing platform automating ICD codes with integration of proper CPT coding of the present invention.

FIG. 2 is a schematic flow chart of the operation of the platform of the present invention in generating a CMS approved audit proof billing.

FIG. 3 is a schematic representation of a personal “dashboard” of a medical practitioner using the questionnaire based medical billing platform automating ICD codes with integration of proper CPT coding of the present invention.

FIG. 4 is a schematic representation of a patient bibliographic information entry of the questionnaire based medical billing platform automating ICD codes with integration of proper CPT coding of the present invention.

FIG. 5 is a schematic representation of a portion of a consult questionnaire of the questionnaire based medical billing platform automating ICD codes with integration of proper CPT coding of the present invention.

FIG. 6 is a schematic representation of a medical history portion of a consult questionnaire of the questionnaire based medical billing platform automating ICD codes with integration of proper CPT coding of the present invention.

FIG. 7 is a schematic representation of an allergy portion of a consult questionnaire of the questionnaire based medical billing platform automating ICD codes with integration of proper CPT coding of the present invention.

FIG. 8 is a schematic representation of an medication portion of a consult questionnaire of the questionnaire based medical billing platform automating ICD codes with integration of proper CPT coding of the present invention.

FIG. 9 is a schematic representation of a portion of a medical necessity questionnaire of the questionnaire based medical billing platform automating ICD codes with integration of proper CPT coding of the present invention.

FIG. 10 is a schematic representation of a portion of a completed operational note template of the questionnaire based medical billing platform automating ICD codes with integration of proper CPT coding of the present invention.

FIG. 11 is a schematic representation of a signature portion of a completed operational note template of the questionnaire based medical billing platform automating ICD codes with integration of proper CPT coding of the present invention.

FIGS. 12A and 12B schematically illustrate a claim or bill prepared with the questionnaire based medical billing platform automating ICD codes with integration of proper CPT coding of the present invention.

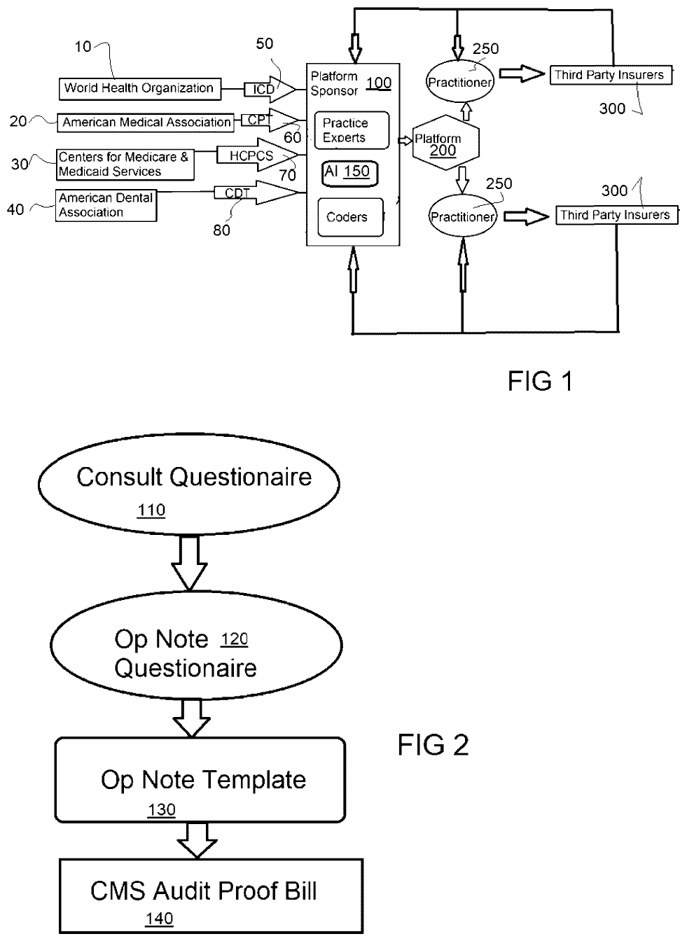

### DESCRIPTION OF THE PREFERRED EMBODIMENTS

A questionnaire based billing platform 200 and associated methodology includes inputting a patient health history, via a consult questionnaire 110, into the platform 200; generating automatically by the platform a list of one or more ICD codes, in an Op note questionnaire 120, based upon the patient health history (from consult questionnaire 110); generating automatically by the platform 200, in the Op note questionnaire 120, a listing of CPT codes associated with the list of one or more ICD codes; selecting on the platform 200, in the Op note questionnaire 120 (also called medical necessity questionnaire 120), by a medical practitioner 250 one or more CPT codes to be performed on the patient from the listing of CPT codes associated with the list of one or more ICD codes; generating an on-line user fillable operative notes template 130 based upon the selected CPT codes, wherein the selected CPT codes are matched with ICD codes; prompting the practitioner 250 to fill out all relevant portions of the on-line user fillable operative notes template 130; obtaining a signature of the practitioner 250 on the on-line user fillable operative notes template 130 following the practitioner 250 filling out all relevant portions of the on-line user fillable operative notes template 130, wherein the practitioner 250 is prevented from providing a signature to complete the template 130 until all relevant portions of the on-line user fillable operative notes template 130 have been properly filled out; and generating a bill by the platform 200 for payers 300 based upon the completed and signed on-line user fillable operative notes template 130.

The medical billing platform 200 of the present invention facilitates medical billing for procedures deemed within the medical scope of the practice of the practitioner 250 and generating billing with appropriate ICD and CPT codes that are CMS approved and audit proof for the payer 300. The platform 200 preferably limits selectable CPT codes to procedures deemed within the medical scope of the practice of medical practitioner 250. In the present application the medical provider 250 can also be described as the health professional, the user, the practitioner, the clinician, the provider, and can also encompass members of the providers practice (e.g. the billing department). These terms are used interchangeably within this application. The medical billing platform 200 of the present invention may also be referenced as the system of the invention, and the terms platform and system are used interchangeably herein. The patient is also called the insured or beneficiary in some cases.

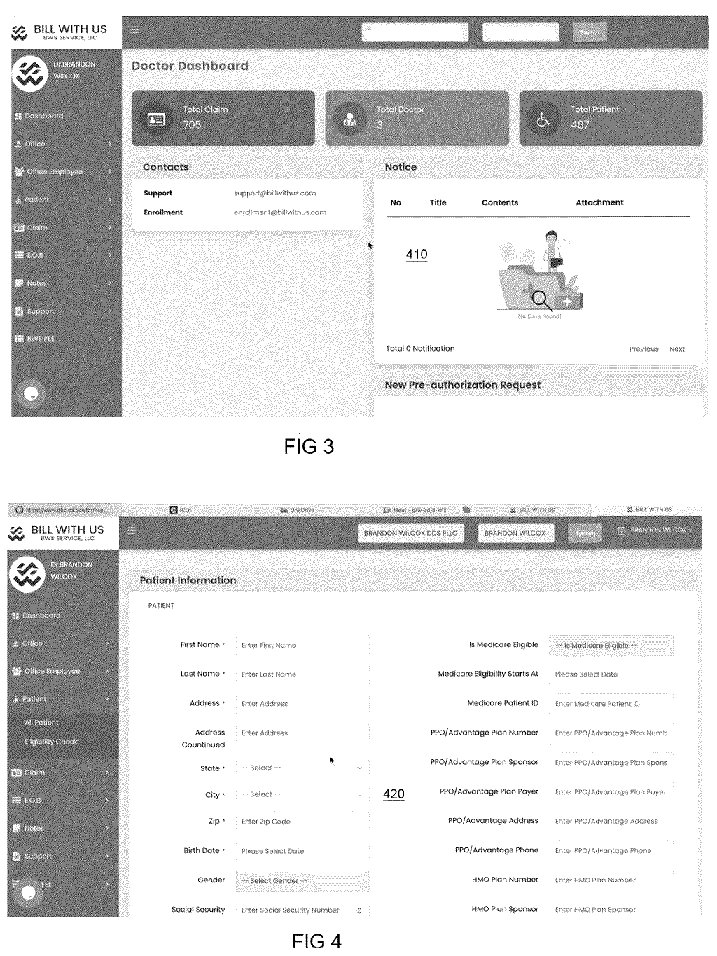

The billing platform 200 of the present invention will generally operate as a software as a service model controlled by the sponsor 100 or vendor 100. Software-as-a-Service (SaaS) is a software support or supply model in which access to the software or platform 200 is provided to practitioners 250 on a subscription basis, with the software being located on external servers rather than on servers located in-house. Software-as-a-Service is typically accessed through a web browser, with users 250 logging into the system using a username and password and accessing via a personal “dashboard” 410 as shown in an example in FIG. 3. Instead of each user or practitioner 250 having to install the software on their office server or computer, the user 250 is able to access the program or platform 200 via the Internet. SaaS is easy to implement, easy to update and debug from the vendor 100, and can be less expensive (or at least have lower up-front costs) since users 250 pay for SaaS as they go instead of purchasing multiple software licenses for multiple computers. For the billing platform 200 of the present invention the SaaS model is convenient as it easily accommodates the use of practice area experts and/or AI 150 at the platform sponsor (or platform vendor 100) to work with the regulatory agencies (WHO 10, AMA 20, CMS 30 and ADA 40 for example) as well as with third party billers 300 (e.g. insurance companies) to create and update the platform 200.

The platform 200 according to the present invention preferably identifies a subset or specialty billing area or scope of practice associated with a set of practitioners 250, like dentistry. Broadly speaking scope of practice refers to those activities that a person licensed to practice as a health professional 250 is permitted to perform, which is increasingly determined by statutes enacted by state legislatures and by rules adopted by the appropriate licensing entity. For example one preferred implementation is dentistry billing and the scope of practice would that associated with dentistry.

In the dental billing field of the present invention the practice area experts at the platform sponsor 100 will be dentists and dental billing experts who can interact with and integrate inputs from the American Dental Association's Current Dental Terminology (CDT 80) (HCPCS Level II dental codes), the American Medical Association's Current Procedural Terminology (CPT 60), the Centers for Medicare & Medicaid Services' Healthcare Common Procedure Coding System (HCPCS level II 70), the World Health Organization's International Classification of Diseases (ICD 50), and billing requirement of third party insurers 300.

The practice area experts can help generate the questionnaires and templates of the platform 200 in language that the dental practitioners understand. The platform 200 can be thought of a translating common dental billing and procedure language into coding acceptable to medical insurance providers 300 and able to survive an audit of such billing.

In addition to practice area experts the vender 100 will naturally have coders to operate and update the platform 200.

More significantly, the implementation of the platform 200 by the vendor can implement AI 150 or machine learning tools to help generate the questionnaires and templates of the platform 200. Broadly speaking AI or artificial intelligence is the theory and development of computer systems capable of performing tasks that historically required human intelligence, such as recognizing speech, making decisions, and identifying patterns. AI is an umbrella term that encompasses a wide variety of technologies, including machine learning, deep learning and natural language processing. At a basic level, machine learning uses algorithms trained on data sets to create machine learning models that allow computer systems to perform tasks like making song recommendations, identifying the fastest way to travel to a destination, or translating text from one language to another. Some of the most common examples of AI in use today include: ChatGPT uses large language models (LLMs) to generate text in response to questions or comments posed to it. Google Translate uses deep learning algorithms to translate text from one language to another. Netflix uses machine learning algorithms to create personalized recommendation engines for users based on their previous viewing history. In the platform 200 of the present invention the platform 200 utilizes AI to generate the questionnaires and templates of the platform 200 based upon input from medical practitioners 250, acceptance of claims by payers 300 and rejections of claims by payers 300 as well as updates changes to ICD codes or CPT codes and the associations between these codes from regulators. The AI 150 of the invention uses algorithms trained on data sets created by the practice area experts to create machine learning models that generate the questionnaires and templates of the platform 200 and then be modified by input from medical practitioners 250, acceptance of claims by payers 300 and rejections of claims by payers 300 as well as updates changes to ICD codes or CPT codes and the associations between these codes from regulators. The AI 150 may be particularly effective at noting the rejections and acceptance of claims from the insurance providers 100 and using these to the questionnaires and templates of the platform 200. Separately the AI 150 can collect data from a number of procedures and outcomes with the platform 200 of the invention and with the volume of data is collected the AI 150 could be used to determine or modify pre-surgery guidelines, surgical guidelines, and post op guidelines to optimize patient outcomes.

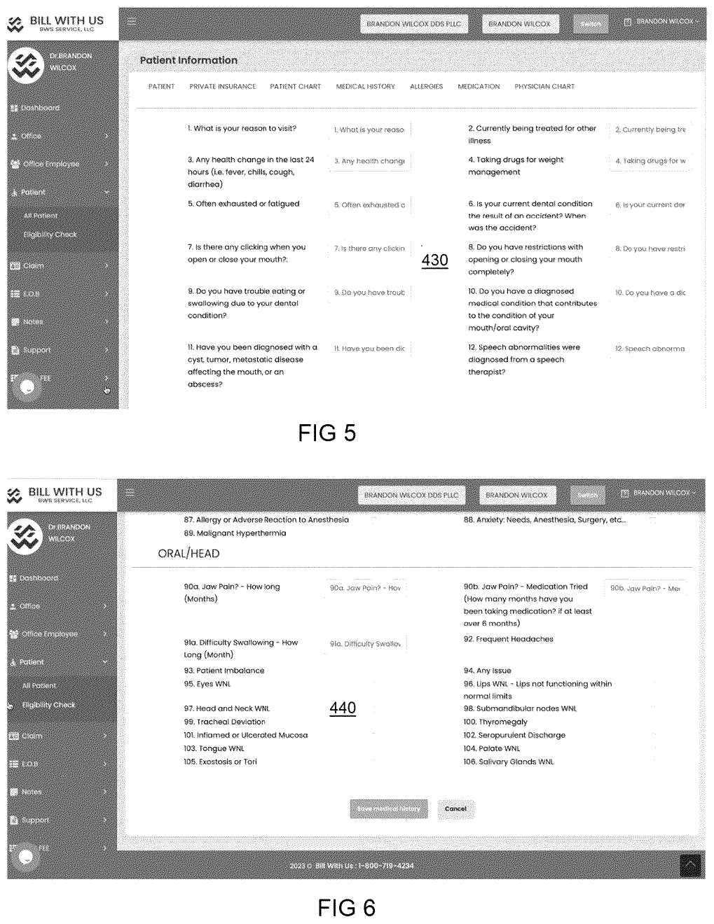

The platform 200 of the present invention from the user 250 perspective begins with entering bibliographic information of a patient into the platform 200 and example of a form 420 for inputting this information is shown in FIG. 4. However substantively, the platform 200 of the present invention from the user 250 perspective begins with entering a health history or consult questionnaire 110, a representative example 430 of which is shown in FIG. 5. FIG. 5 shows a form with a leading question that is not in a yes or no format, namely the patient's description of the reason for the visit, which is a standard intake question. The substance of the consult questionnaire 110 is a series of yes or no questions configured in the common procedural language of the subset of the medical professional (e.g. dentistry, orthopedics, etc.). FIG. 6 schematically shows a medical history portion 440 of the questionnaire 110, again the medical history template questions are specific to the field or area of practice of the medical professional. The practitioner can easily check the yes for those that apply for the patient. In the dentistry example this questionnaire 110 is generally following Current Dental Terminology and other common dental descriptions whereby the question are in a format familiar to the Dentists.

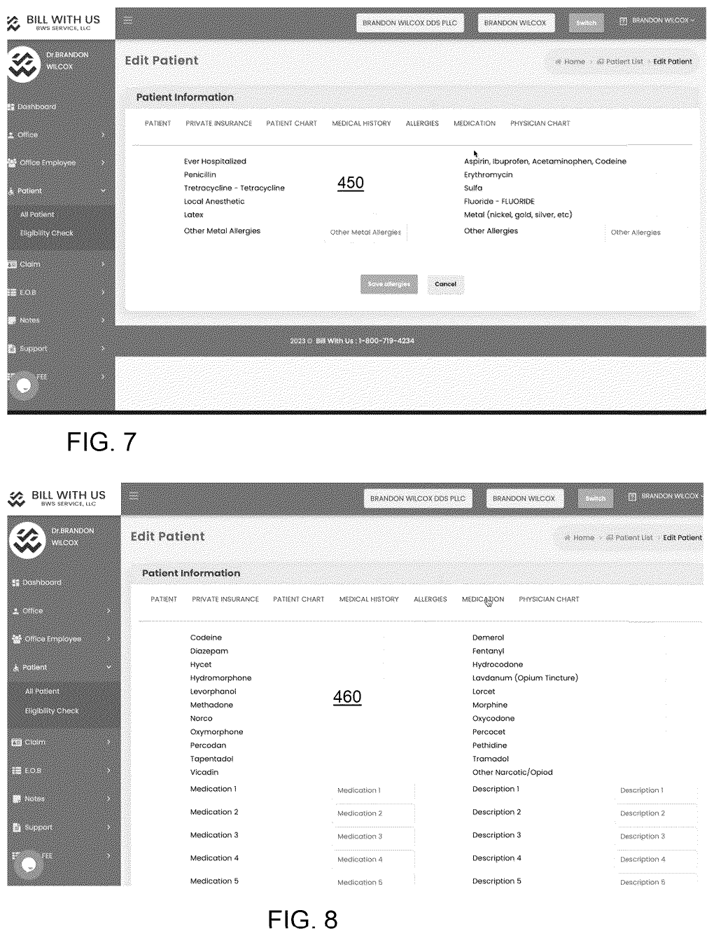

FIG. 7 is a schematic representation of an allergy portion 450 of a consult questionnaire 110 of the questionnaire based medical billing platform automating ICD codes with integration of proper CPT coding of the present invention. The allergy portion 450 is also illustrative of another aspect of the generally yes/no questionnaire 110. The questionnaire 110 includes the yes or no question of other allergies, but if yes is supplied the practitioner would need to enter the specifics. Other yes/no questions may require additional input to complete the record. FIG. 8 is a schematic representation of an medication portion 460 of a consult questionnaire 110 of the questionnaire based medical billing platform automating ICD codes with integration of proper CPT coding of the present invention. Like the allergy portion 450, the medication portion is also illustrative of selectively adding descriptive text to complete a yes answer for “other medications”. The goal of the system 200 is to include as many standard or common medications as possible in the yes/no format. The AI 150 can be helpful to tailor the questionnaire over time to most efficiently list the relevant allergies and medications and the like.

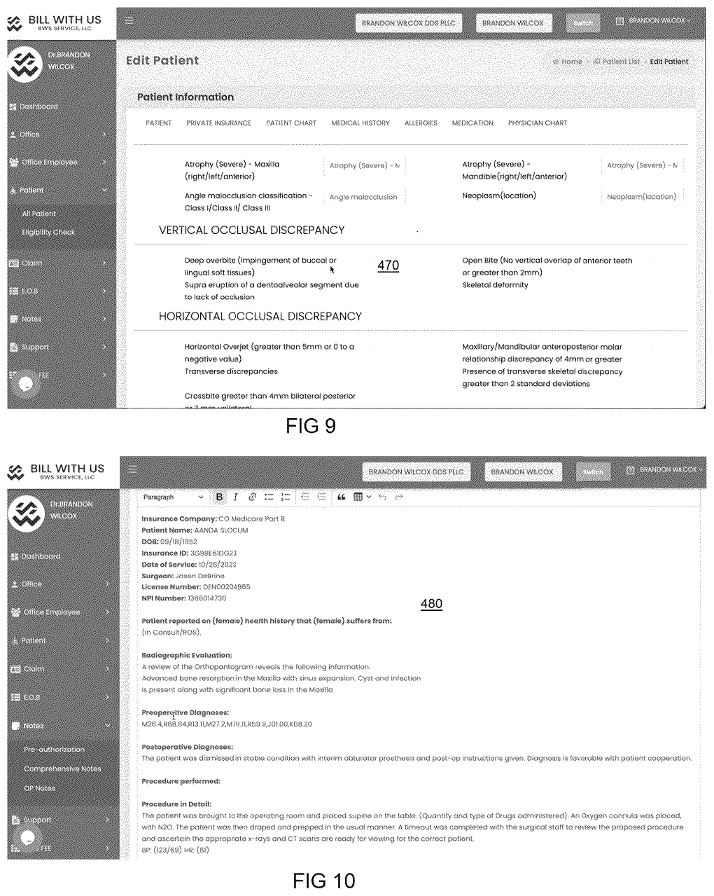

The positive or yes answers to the consult questionnaire 110 generate and will, in short, populate the Op note questionnaire 120 or medical necessity questionnaire 120 with associated and proper ICD codes (ICD-10 and/or ICD-11) for medical necessity as represented schematically in screen 470 in FIG. 9. More specifically the platform 200 generates a list of one or more ICD codes based upon the patient health history or completed questionnaire 110 and the platform 200 automatically generates a listing of CPT codes associated with the list of one or more ICD codes.

“Medical necessity” is somewhat difficult to precisely define, with as many different interpretations as there are payers; however, most definitions incorporate the idea that healthcare services must be “reasonable and necessary” or “appropriate,” given a patient's condition and the current standards of clinical practice. Yet typically, the decision as to whether services are medically necessary is made by someone who has never seen the patient. Medicare defines “medical necessity” as services or items reasonable and necessary for the diagnosis or treatment of illness or injury or to improve the functioning of a malformed body member. CMS has the power under the Social Security Act to determine, on a case-by-case basis, if the method of treating a patient is reasonable and necessary. For all payers 300 and insurance plans, even if a service is reasonable and necessary, coverage may be limited if the service is provided more frequently than allowed under a national coverage policy, a local medical policy, or a clinically accepted standard of practice. Claims for services deemed to be not medically necessary will typically be denied. Further, if Medicare (or any other payer 300) pay for services that they later determine to be not medically necessary, they may demand that those payments be refunded (often with interest) from the practitioner 250. If a pattern of such claims can be established, and the provider 250 knows (or should know) that the services reported were not medically necessary, the provider 250 may face monetary penalties, exclusion from Medicare program, and possible criminal prosecution. The platform 200 aims to minimize these possibilities for practitioners 250.

From a clinical perspective, medical necessity is determined by the provider 250 based on evidence-based medical data. This data may be used to order further testing to diagnose a patient's condition or provide additional procedures to treat a patient's condition. The op note questionnaire 120 or medical necessity questionnaire 120 (a portion of which is shown in FIG. 9 at screen 470) allows the practitioner 250 to select on the platform 200 one or more CPT codes to be performed on the patient from the listing of CPT codes associated with the list of one or more ICD codes.

Often when a procedure or test is ordered, the provider 250 often must first get approval from the patient's payer 300 before performing the test or procedure. In giving this approval, the payer 300 is saying the test or procedure meets their established medical necessity criteria. It must be noted that prior authorization is not always a guarantee of payment, however, but accurate and documented prior authorizations will greatly increase the likelihood of payment. The platform 200 easily allows for including the step of submitting pre-authorizations to payers 300, wherein the pre-authorizations to payers 300 is based upon the selected CPT codes, wherein the selected CPT codes are matched with ICD codes.

From an insurance or payer's 300 perspective, medical necessity is determined by either the diagnosis code(s) and/or clinical condition(s) that are defined in the payer's policy. The pre-approval process typically involves submitting to the payer 300: the patient's diagnosis; and the procedure to be performed. A provider 250 generally should also include: the severity of the diagnosis; the risk of not performing the procedure; and any diagnostic studies or interventions tried previously. The platform 200 easily formats and accommodates these requirements. The AI 150 can adapt and adjust the pre-approvals prepared by the system based upon accepted and rejected pre-approval requests from earlier patients.

The preparation of prior authorization may simply be used for patient information and consent as a pre-procedure description of the patient's diagnosis, the relevant procedures to be performed; the severity of the diagnosis; the risk of not performing the procedure; and any diagnostic studies or interventions tried previously. The platform allows the practitioner 250 to obtain a signature and/or possibly a biometric input (e.g., a fingerprint, retinal scan, face scan, or other unique biometric identifier) from the patient on the pre-procedure description and/or the prior authorization. The signature and/or biometric will minimize opportunities of insurance fraud and illustrate informed consent of the patient.

As noted the platform 200 of the present invention includes templates to allow the practitioner to easily incorporate the severity of the diagnosis; the risk of not performing the procedure and can include appropriate prompts for the needed support. In addition the system 200 allows uploading and automatic inputting of patient history, typically from an electronic health record of the patient. Electronic health records can be considered digital versions of a patients file, but actually are real time, patient centered records that make information available instantly and securely to all authorized users. As a representative example the platform 200 easily uploads Radiology reports, and any physician referrals, lab tests, blood work. This includes pathology reports post-surgery.

It is important for the physician 250, coder, biller, and insurance company 300 to all be on the same page when it comes to medical necessity. A provider 250 may feel specific procedures or tests are medically necessary for a patient, but the insurance company 300 can also make that determination based on their clinical policies. Problems ensue when medical necessity is defined differently by the two parties. The platform 200 of the present invention minimizes the likelihood of differences by coordinating the coding for the diagnosis with the procedure as well as properly documenting the case in question.

It is important to reiterate that the health history or consult questionnaire 110 of the system 200 of the invention is designed to be comprehensive and in the language most associated with the subset specialty. Following the simple yes/no health history or consult questionnaire 110 the platform 200 of the present invention will populate ICD 10 codes for the diagnosis of medical necessity.

With the proper ICD 10 codes identified the system 200 will generate an operation note questionnaire 130 associated with the identified ICD 10 codes. The operational note questionnaire 130 is designed to interact with the professional 250 with inquiries and prompts that are used to generate and complete the appropriate operational note template. Again the operational note questionnaire 130 will preferably largely be in yes no questions in language most familiar with the specialty. Any patient information and ICD 10 codes previously identified are automated downfield in all op notes.

The operation note (often termed the “op note”) is the completed operational note template 130 with signature and is a vital document that records exactly what procedures were performed upon a patient, what was found and/or occurred during the procedures, and what are the post-procedure instructions for the patient from the medical practitioner. It is generally considered to provide part of the “medicolegal” record of a patient's care during their stay. As such it is a key document and must contain certain information. The platform 200 of the invention facilitates the completeness of this document.

Operative Note templates 130 are generated from Op note questionnaire 120, that includes all Patient info and ICD-10 codes upfield that includes the generation of correct possible CPT codes for Dentist/physician 250 to choose from that are billable. This selection process in the questionnaire 120 in the platform 200 of the invention generates OP note templates 130 that have coding and fillable fields which have been approved by CMS and Medicare Advantage audits. FIG. 10 is a schematic representation of a portion 480 of a completed operational note template 130 of the questionnaire based medical billing platform 200 automating ICD codes with integration of proper CPT coding of the present invention.

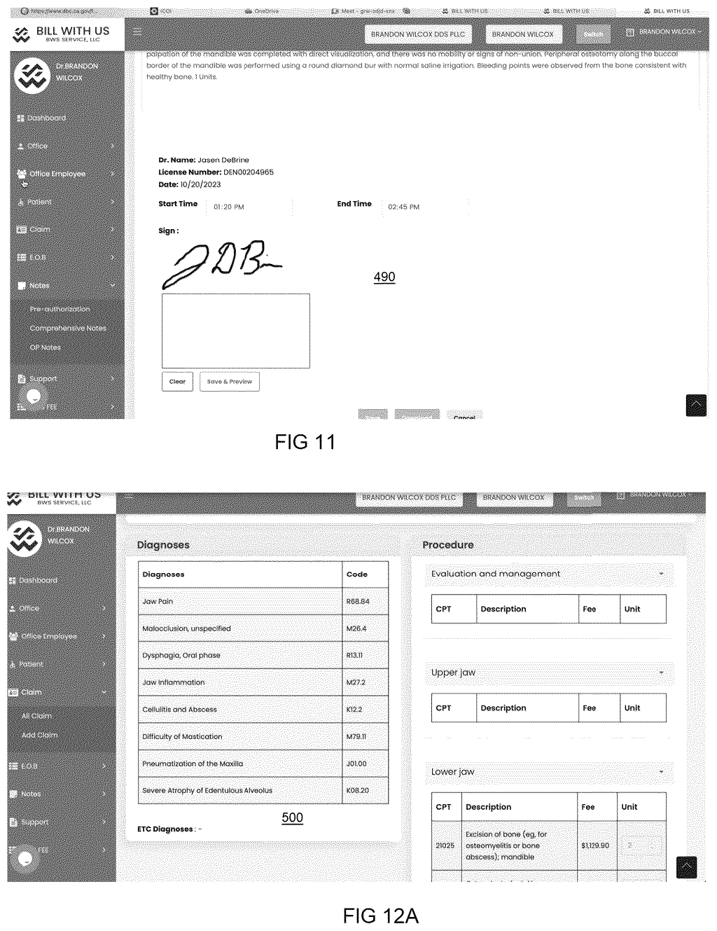

The platform 200 obtains a signature of the medical practitioner 250 on the on-line user fillable operative notes template 130 following the practitioner 250 filling out all relevant portions of the on-line user fillable operative notes template 130, wherein the medical practitioner 200 is prevented from providing a signature to complete the on-line user fillable operative notes template 130 until all relevant portions of the on-line user fillable operative notes template 130 have been properly filled out. FIG. 11 is a schematic representation of a signature portion 490 of a completed operational note template 130 of the questionnaire based medical billing platform 200 automating ICD codes with integration of proper CPT coding of the present invention.

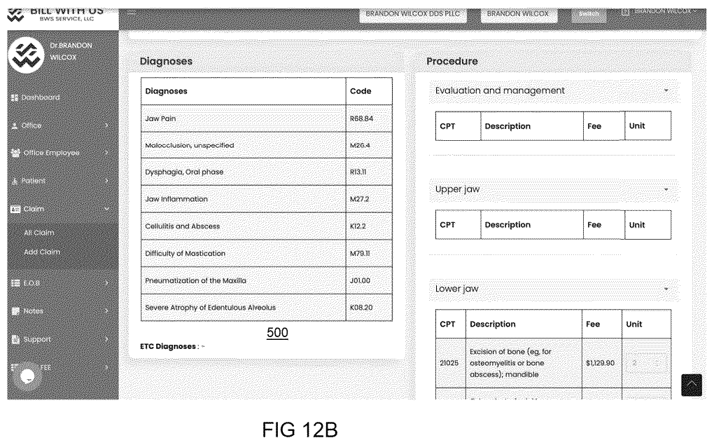

The platform 200 generates a bill, or claim, based upon the completed and signed on-line user fillable operative notes template 130 (now the op notes). FIGS. 12A and 12B schematically illustrate a claim or bill 500 prepared from a completed and signed on-line user fillable operative notes template 130. The billing can be sent to the payers 300 for processing and acceptances (or rejections and the reasons therefore) can be used to build the system through the AI 150. The platform 200 of the present invention can generate billing 500 and operational notes from the completed templates 130 which the platform 200 requires to be signed by dentist/physician 250. The platform 200 will include e-signature (a signature in electronic form) or the platform 200 can accept “wet signatures” that are digitized/scanned into the system.

As discussed above, the platform 200 allows the practitioner 250 to easily submit pre-authorizations to payers 300 in formats better suited for review and approval. Further the platform is configured to upload Explanation of Benefits (EOBs) into the platform 200. An explanation of benefits (EOB) is the insurance company's written explanation regarding a claim or authorization, showing what they paid or will pay and what the patient must pay. The document is sometimes accompanied by a benefits check, but it's more typical for the insurer to send payment directly to the medical provider. The EOB is not a bill, although it will explain any charges that the patient still owes or may have already paid (in the form of a copay at the time the medical care was received, for example), or would owe if a preauthorized procedure is selected. Pre-authorization and EOB integration allow for qualification of patients and a careful review of patients options including their costs associated with each plan of care. This can remove uncertainty for patients and greatly improve patient care and outcomes. The integration of EOB will further simplify bookkeeping for the practitioner by more easily tracking the charges and categorization of such charges (e.g. unreimbursed, reimbursed, allowable, billable, etc.)

The platform 200 in summary includes simple questionnaires to generate appropriate templates that allow a practitioner such as a dentist to give predetermined input and will output final op notes acceptable to medical insurances. The platform 200 simplifies submission of claims for reimbursement.

It will be apparent to those of ordinary skill in the art that various changes may be made to the present invention without departing from the spirit and scope thereof.

What is claimed is:

1. A questionnaire based billing platform methodology comprising the steps of:
i) Inputting a patient health history into the platform;
ii) Generating automatically by the platform a list of one or more ICD codes based upon the patient health history;
iii) Generating automatically by the platform a listing of CPT codes associated with the list of one or more ICD codes;
iv) Selecting on the platform by a medical practitioner one or more CPT codes to be performed on the patient from the listing of CPT codes associated with the list of one or more ICD codes;
v) Generating an on-line user fillable operative notes template based upon the selected CPT codes, wherein the selected CPT codes are matched with ICD codes;
vi) Prompting the medical practitioner to fill out all relevant portions of the on-line user fillable operative notes template;
vii) Obtaining a signature of the medical practitioner on the on-line user fillable operative notes template following the practitioner filling out all relevant portions of the on-line user fillable operative notes template, wherein the medical practitioner is prevented from providing a signature to complete the on-line user fillable operative notes template until all relevant portions of the on-line user fillable operative notes template have been properly filled out; and
viii) Generating a bill by the platform based upon the completed and signed on-line user fillable operative notes template.

2. The questionnaire based billing platform methodology according to claim 1, wherein the inputting a patient health history into the platform is via an on-line consult questionnaire input form having a series of yes or no questions.

3. The questionnaire based billing platform methodology according to claim 2, wherein the on-line consult questionnaire is in a common procedural language for procedures deemed within the medical scope of the practice of medical practitioner.

4. The questionnaire based billing platform methodology according to claim 1, wherein the platform limits selectable CPT codes to procedures deemed within the medical scope of the practice of medical practitioner.

5. The questionnaire based billing platform methodology according to claim 1, the completed and signed on-line user fillable operative notes template forms a signed record of i) procedures performed upon the patient, ii) findings of medical note discovered during the procedures, iii) occurrences of medical note during the medical procedure, and iv) post-procedure instructions for the patient from the medical practitioner.

6. The questionnaire based billing platform methodology according to claim 1, further including the step of submitting pre-authorizations to payers wherein the pre-authorizations to payers is based upon the selected CPT codes, wherein the selected CPT codes are matched with ICD codes.

7. The questionnaire based billing platform methodology according to claim 1, further including the step of obtaining a signature or biometrics from the patient on a pre-procedure description within the platform prior performing procedures on the patient.

8. The questionnaire based billing platform methodology according to claim 1, further including the step of uploading an Explanation of Benefits into the platform for the patient wherein the Explanation of Benefits is payer's written explanation regarding a claim or authorization showing at least what the payer paid or will pay and what the patient paid or will pay.

9. The questionnaire based billing platform methodology according to claim 1, wherein the on-line user fillable operative notes template based upon the selected CPT codes further includes any necessary Healthcare Common Procedure Coding System codes associated with the procedures.

10. The questionnaire based billing platform methodology according to claim 1, wherein the platform utilizes AI to generate the questionnaires and templates of the platform based upon input from medical practitioners, acceptance of claims by payers and rejections of claims by payers.

11. The questionnaire based billing platform methodology according to claim 1, wherein the platform automatically updates changes to ICD codes or CPT codes and the associations between these codes.

12. An on-line questionnaire based billing platform comprising:
i) An online patient health history input form for each patient on the platform;
ii) An automatically generated listing of one or more ICD codes based upon each patient health history;
iii) An automatically generated listing of CPT codes associated with the list of one or more ICD codes;
iv) Select CPT codes to be performed on the patient which are selected by the medical practitioner from the listing of CPT codes associated with the list of one or more ICD codes;
v) An on-line user fillable operative notes template generated by the platform based upon the selected CPT codes, wherein the selected CPT codes are matched with ICD codes;
vi) Wherein the system is configured to prompt the medical practitioner to fill out all relevant portions of the on-line user fillable operative notes template;
vii) Wherein the system is configured to obtain a signature of the medical practitioner on the on-line user fillable operative notes template following the practitioner filling out all relevant portions of the on-line user fillable operative notes template, wherein the medical practitioner is prevented from providing a signature to complete the on-line user fillable operative notes template until all relevant portions of the on-line user fillable operative notes template have been properly filled out; and
viii) a bill generated by the platform based upon the completed and signed on-line user fillable operative notes template.

13. The questionnaire based billing platform according to claim 12, the patient health history input form is in a common procedural language for procedures deemed within the medical scope of the practice of medical practitioner.

14. The questionnaire based billing platform according to claim 12, wherein the platform limits selectable CPT codes to procedures deemed within the medical scope of the practice of medical practitioner.

15. The questionnaire based billing platform according to claim 12, wherein the completed and signed on-line user fillable operative notes template forms a signed record of i) procedures performed upon the patient, ii) findings of medical note discovered during the procedures, iii) occurrences of medical note during the medical procedure, and iv) post-procedure instructions for the patient from the medical practitioner.

16. The questionnaire based billing platform according to claim 1, further including pre-authorizations to payers generated by the platform.

17. The questionnaire based billing platform according to claim 16, wherein the pre-authorizations to payers is based upon the selected CPT codes, wherein the selected CPT codes are matched with ICD codes.

18. The questionnaire based billing platform according to claim 12, further including the Explanation of Benefits uploaded into the platform for the patient wherein the Explanation of Benefits is payer's written explanation regarding a claim or authorization showing at least what the payer paid or will pay and what the patient paid or will pay.

19. The questionnaire based billing platform according to claim 12, wherein the on-line user fillable operative notes template based upon the selected CPT codes further includes any necessary Healthcare Common Procedure Coding System codes associated with the procedures.

20. The questionnaire based billing platform according to claim 1, wherein the platform utilizes AI to generate the questionnaires and templates of the platform based upon input from medical practitioners, acceptance of claims by payers and rejections of claims by payers.
